(19) United States
(12) Patent Application Publication
(10) Pub. No.: US 2026/0265165 A1
(43) Pub. Date: Sep. 10, 2026

(54) PROCESSES FOR CONVERTING OLEFINS IN PLASTIC WASTE PYROLYSIS OIL TO ALKENE PRODUCTS

(71) Applicant: Dow Global Technologies LLC, Midland, MI (US)

(72) Inventors: Rafael Huacuja, Rosharon, TX (US); Ivan A. Konstantinov, Manvel, TX (US); Edmund M. Carnahan, Pearland, TX (US); Sean W. Ewart, Pearland, TX (US); Mari S. Rosen, Houston, TX (US)

(73) Assignee: Dow Global Technologies LLC, Midland, MI (US)

(21) Appl. No.: 18/875,495

(22) PCT Filed: Jun. 14, 2023
(86) PCT No.: PCT/US2023/068416

§ 371 Date: Dec. 16, 2024

Related U.S. Application Data
(60) Provisional application No. 63/353,322, filed on Jun. 17, 2022.

(51) Int. Cl. C07C 6/04

(52) U.S. Cl. CPC C07C 6/04

(57) ABSTRACT

This disclosure relates to processes for converting olefins from plastic waste pyrolysis oil to at least an alkene product. The processes comprise contacting the plastic waste pyrolysis oil with two or more catalyst components in a reactor, the reactor comprising an alkene reactant. The two or more catalyst components comprise a metathesis catalyst component and an isomerization catalyst component. Contacting causes at least a portion of the olefins in the plastic waste pyrolysis oil, or products derived therefrom, to undergo metathesis reactions and isomerization reactions to produce an effluent comprising at least the alkene product.

### CROSS-REFERENCE TO RELATED APPLICATIONS

This application claims the benefit of U.S. Provisional Application Ser. No. 63/353,322 filed Jun. 17, 2022, the entire disclosure of which is hereby incorporated herein by reference.

### BACKGROUND

### Field

The present disclosure relates to chemical processing. In particular, the present disclosure relates to processes for converting olefins in pyrolysis oil, such as plastic waste pyrolysis oil, into smaller desirable hydrocarbon products.

### Technical Background

For a number of industrial applications, hydrocarbons are used, or are starting materials used, to produce plastics, fuels, and various downstream chemicals. Such hydrocarbons include alkenes, such as ethene, propene and butenes (also commonly referred to as ethylene, propylene, and butylenes, respectively). A variety of processes for producing these lower hydrocarbons have been developed, including petroleum cracking and various synthetic processes. Pyrolysis of plastic waste can be used to recycle the plastic waste. Pyrolysis of plastic waste can form pyrolysis oil. The pyrolysis oil can be used as a feedstock to make more desirable hydrocarbons. However, additional processing is first needed to remove olefinic compounds in the pyrolysis oil. Conventional efforts for removing olefins from pyrolysis oil include hydroprocessing. These processes are energy inefficient, and require high operating temperatures. New methods for processing waste plastic pyrolysis oil are needed.

### SUMMARY

Embodiments of the present disclosure address these and other needs by providing processes for converting olefins in plastic waste pyrolysis oil to smaller alkene products. The processes described herein may enable two or more catalyst components in a reactor system to conduct a plurality of different chemical reactions, such as combinations of metathesis and isomerization for producing alkene products from plastic waste pyrolysis oil and an alkene reactant, for example.

According to one or more other aspects of the present disclosure, a process for converting olefins in plastic waste pyrolysis oil to at least an alkene product of chemical formula CₘH₂ₘ, the process comprising contacting the plastic waste pyrolysis oil with two or more catalyst components in a reactor, the reactor comprising an alkene reactant of chemical formula CₙH₂ₙ, where m is an integer from 3 to 20 and n is an integer from 2 to 20. The two or more catalyst components comprise a metathesis catalyst component and an isomerization catalyst component. Contacting causes at least a portion of the olefins in plastic waste pyrolysis oil, or products derived therefrom, to undergo metathesis reactions and isomerization reactions to produce an effluent comprising at least the alkene product of chemical formula CₘH₂ₘ.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### BRIEF DESCRIPTION OF THE DRAWING

FIG. 1 is ¹H nuclear magnetic resonance (NMR) spectra, in accordance with one or more embodiments of the present disclosure.

FIG. 2 is ¹H nuclear magnetic resonance (NMR) spectra, in accordance with one or more embodiments of the present disclosure.

FIG. 3 is a bar graph, in accordance with one or more embodiments of the present disclosure.

FIG. 4 is a bar graph, in accordance with one or more embodiments of the present disclosure.

FIG. 5 is a depiction of a catalytic reaction and catalyst compositions, in accordance with one or more embodiments of the present disclosure.

### DETAILED DESCRIPTION

Some conventional processes for converting olefins in pyrolysis oil may use energy intensive and inefficient processes, such as hydroprocessing to form aliphatics, which are then cracked to form lighter products, which can increase the capital cost of operation and have an increased CO₂ footprint. In contrast, processes disclosed herein can enable tandem catalysis of olefins in plastic waste pyrolysis oil to directly form alkene products by contacting the plastic waste pyrolysis oil with mutually compatible catalyst components to produce the desired alkene products. The catalytic breaking of higher weight olefins under mild reaction conditions provides an advantageous and sustainable alternative for the production of hydrocarbon feedstock, monomers or other useful chemicals.

Reference will now be made in detail to embodiments of processes for converting olefins in plastic waste pyrolysis oil to alkene products in a reactor. As used herein, “plastic waste pyrolysis oil” refers to a mixture of products derived from the pyrolysis of plastic waste. The process of pyrolysis of plastic waste can produce a mixture of liquid oil, gases, waxes, and char. While the liquid oil is referred to as pyrolysis oil, in embodiments described herein “pyrolysis oil” refers to the liquid oil, which may include any of the products from the process of pyrolysis such as waxes. In embodiments, the pyrolysis oil comprises a mixture of at least paraffins, olefins, naphthenes, and aromatics. In embodiments, the plastic waste pyrolysis oil can be processed before using in the reactor, such as but not limited to purification. For instance, in embodiments, the plastic waste pyrolysis oil can be purified through various techniques, including but not limited to filtration before the plastic waste pyrolysis oil contacts the two or more catalyst compositions in the reactor. In embodiments, the plastic waste pyrolysis oil can be purified by any methods known in the art, such as passing through a column comprising silica, alumina, or combinations thereof. In embodiments, one or more additives can be included in the pyrolysis oil.

In embodiments, the plastic waste pyrolysis oil can comprise olefins in an amount of from 20 weight percent (wt. %) to 100 wt. %, based on the total weight of the plastic waste pyrolysis oil. For instance, the plastic waste pyrolysis oil can comprise olefins in an amount of from 20 wt. % to 90 wt. %, from 20 wt. % to 80 wt. %, from 20 wt. % to 70 wt. %, from 20 wt. % to 60 wt. %, from 20 wt. % to 50 wt. %, from 20 wt. % to 40 wt. %, from 30 wt. % to 100 wt. %, from 30 wt. % to 90 wt. %, from 30 wt. % to 80 wt. %, from 30 wt. % to 70 wt. %, from 30 wt. % to 60 wt. %, from 30 wt. % to 50 wt. %, from 40 wt. % to 100 wt. %, from 40 wt. % to 90 wt. %, from 40 wt. % to 80 wt. %, from 40 wt. % to 70 wt. %, from 40 wt. % to 60 wt. %, from 50 wt. % to 80 wt. %, from 50 wt. % to 70 wt. %, from 50 wt. % to 60 wt. %, from 60 wt. % to 80 wt. %, or from 60 wt. % to 70 wt. %.

In embodiments, the plastic waste pyrolysis oil can comprise mono-olefins in an amount of from 20 weight percent (wt. %) to 100 wt. %, based on the total weight of the plastic waste pyrolysis oil. For instance, the plastic waste pyrolysis oil can comprise olefins in an amount of from 20 wt. % to 90 wt. %, from 20 wt. % to 80 wt. %, from 20 wt. % to 70 wt. %, from 20 wt. % to 60 wt. %, from 20 wt. % to 50 wt. %, from 20 wt. % to 40 wt. %, from 30 wt. % to 100 wt. %, from 30 wt. % to 90 wt. %, from 30 wt. % to 80 wt. %, from 30 wt. % to 70 wt. %, from 30 wt. % to 60 wt. %, from 30 wt. % to 50 wt. %, from 40 wt. % to 100 wt. %, from 40 wt. % to 90 wt. %, from 40 wt. % to 80 wt. %, from 40 wt. % to 70 wt. %, from 40 wt. % to 60 wt. %, from 50 wt. % to 80 wt. %, from 50 wt. % to 70 wt. %, from 50 wt. % to 60 wt. %, from 60 wt. % to 80 wt. %, or from 60 wt. % to 70 wt. %.

In embodiments, the plastic waste pyrolysis oil can comprise diolefins in an amount of from 0 weight percent (wt. %) to 10 wt. %, based on the total weight of the plastic waste pyrolysis oil. For instance, the plastic waste pyrolysis oil can comprise diolefins in an amount of from 0 wt. % to 10 wt. %, from 0 wt. % to 5 wt. %, from 1 wt. % to 10 wt. %, from 1 wt. % to 5 wt. %, or from 5 wt. % to 10 wt. %.

In embodiments, the plastic waste pyrolysis oil can comprise paraffins in an amount of from 20 weight percent (wt. %) to 60 wt. %, based on the total weight of the plastic waste pyrolysis oil. For instance, the plastic waste pyrolysis oil can comprise paraffins in an amount of from 20 wt. % to 50 wt. %, from 20 wt. % to 40 wt. %, from 30 wt. % to 60 wt. %, from 30 wt. % to 50 wt. %, from 40 wt. % to 60 wt. %, or from 40 wt. % to 50 wt. %.

In embodiments, the plastic waste pyrolysis oil can comprise naphthenes in an amount of from 10 wt. % to 30 wt. %, based on the total weight of the plastic waste pyrolysis oil. For instance, the plastic waste pyrolysis oil can comprise naphthenes in an amount of from 10 wt. % to 25 wt. %, from 10 wt. % to 20 wt. %, from 10 wt. % to 15 wt. %, from 15 wt. % to 30 wt. %, from 15 wt. % to 25 wt. %, from 15 wt. % to 20 wt. %, or from 20 wt. % to 30 wt. %.

In embodiments, the plastic waste pyrolysis oil can comprise aromatics in an amount of from 5 wt. % to 15 wt. %, based on the total weight of the plastic waste pyrolysis oil. For instance, the plastic waste pyrolysis oil can comprise naphthenes in an amount of from 5 wt. % to 12 wt. %, from 5 wt. % to 10 wt. %, from 10 wt. % to 15 wt. %, or from 10 wt. % to 12 wt. %.

In embodiments, the plastic waste pyrolysis oil can comprise Cl, Si, O, and N in an amount less than 2 wt. %, less than 1 wt. %, less than 0.8 wt. %, or less than 0.6 wt. %.

In embodiments, the plastic waste pyrolysis oil can have a density of from 0.64 g/cm³ to 0.95 g/cm³. For instance, in embodiments, the plastic waste pyrolysis oil can have a density of from 0.64 g/cm³ to 0.90 g/cm³, from 0.64 g/cm³ to 0.85 g/cm³, from 0.64 g/cm³ to 0.80 g/cm³, from 0.64 g/cm³ to 0.75 g/cm³, 0.64 g/cm³ to 0.70 g/cm³, from 0.75 g/cm³ to 0.95 g/cm³, from 0.75 g/cm³ to 0.90 g/cm³, from 0.75 g/cm³ to 0.85 g/cm³, from 0.75 g/cm³ to 0.80 g/cm³, from 0.80 g/cm³ to 0.90 g/cm³, or from 0.80 g/cm³ to 0.95 g/cm³. In embodiments, the plastic waste pyrolysis oil can have a viscosity of from 0.5 centipoise (cp) to 1000 cp.

In embodiments, the reactor comprises an alkene reactant. In embodiments, the alkene reactant has a chemical formula of CₙH₂ₙ, where n is an integer from 2 to 20. For example, the alkene reactant can have a chemical formula of CₙH₂ₙ, where n is an integer from 2 to 15, from 2 to 10, from 2 to 5, from 2 to 4, or from 2 to 3. In embodiments, the alkene reactant can comprise ethylene, propylene, butenes, pentenes, or combinations thereof. In embodiments, the alkene reactant can be selected from the group consisting of ethylene, propylene, butenes, pentenes, and combinations thereof. In embodiments, the alkene reactant can comprise ethylene. In embodiments, the alkene reactant can consist essentially of or consist of ethylene. In embodiments, the alkene reactant can comprise ethylene and butenes. In embodiments, the alkene reactant can consist essentially of or consist of ethylene and butenes.

In embodiments, the plastic waste pyrolysis oil can be contacted with two or more catalyst components in a reactor. In embodiments, the plastic waste pyrolysis oil can be contacted with three or more catalyst components in a reactor. As used herein, “catalyst components” refers to any substance which increases the rate of a specific chemical reaction. Catalyst components and the catalyst compositions made with the catalyst components described in this disclosure may be utilized to promote various reactions, such as, but not limited to, dehydrogenation, metathesis, isomerization, or combinations of these. In embodiments, a catalyst composition can include at least one catalyst component or at least two catalyst components. As used herein, “catalyst composition” refers to a solid particulate comprising at least one catalyst component. The catalyst composition can further comprise a catalyst support material.

In embodiments the catalyst components can include a metathesis catalyst component and an isomerization catalyst component. In embodiments the catalyst components can include a dehydrogenation catalyst component, a metathesis catalyst component, and an isomerization catalyst component. Without intending to be bound by any particular theory, it is believed that the metathesis catalyst, in the presence of the alkene reactant, can break the carbon chain of the olefins in the plastic waste pyrolysis oil to produce two products that each have a terminal unsaturation, and further metathesis of the terminally unsaturated intermediate product with the alkene reactant may be unproductive to further break the carbon chain. It is believed that the isomerization catalyst component can convert the terminal unsaturation to an internal unsaturation, and the isomerized product can be further broken into two products in the presence of the metathesis catalyst component and the alkene reactant. It is believed that the products derived from the olefins in the plastic waste pyrolysis oil that contact both the metathesis catalyst component and the isomerization catalyst component in the presence of the alkene reactant can continue to cycle between metathesis and isomerization reactions to produce smaller alkene products, such as compounds of chemical formula CₘH₂ₘ, where m is an integer from 3 to 20, for instance, propylene. In embodiments, the reaction time can be increased to produce an effluent comprising smaller alkene products, as increased reaction time will allow additional metathesis and isomerization reaction cycles. In embodiments, where the alkene reactant comprises ethylene, the metathesis catalyst component can cause ethenolysis of the plastic waste pyrolysis oil, or products derived therefrom.

In embodiments, the metathesis catalyst component in combination with the alkene reactant, such as ethylene, can be operable to break the olefin chain into two species. In embodiments, the metathesis catalyst component can break alkene products derived from the plastic waste pyrolysis oil. In embodiments, the metathesis catalyst component can include one or more elements selected from International Union of Pure and Applied Chemistry (IUPAC) groups 5-10. In embodiments, the metathesis catalyst component can comprise rhenium, ruthenium, tungsten, molybdenum, vanadium, or combinations thereof. In embodiments, the metathesis catalyst component can be selected from the group consisting of rhenium, ruthenium, tungsten, molybdenum, vanadium, and combinations thereof.

In embodiments, the isomerization catalyst component can be operable to move an unsaturation on olefins, or an unsaturation on products derived therefrom, from one position on the backbone to a different position. For instance, in embodiments, the isomerization catalyst component can move an unsaturation in a terminal position of the olefins to an internal position. In embodiments, the isomerization catalyst component can include one or more elements selected from International Union of Pure and Applied Chemistry (IUPAC) groups 5-10. In embodiments, the isomerization catalyst component can comprise alumina, silica, iridium, palladium, ruthenium or combinations thereof. In embodiments, the isomerization catalyst component can be selected from the group consisting of alumina, silica, iridium, palladium, ruthenium, and combinations thereof. In embodiments, the isomerization catalyst component can include modified alumina, modified silica, or combinations thereof.

In embodiments, the dehydrogenation catalyst component can be operable to cause olefins, or products derived therefrom, to have additional unsaturations. Without intending to be bound by any particular theory, it is believed that the additional unsaturations caused by the dehydrogenation catalyst component can speed up the occurrence of the metathesis and/or isomerization reactions of the olefins, or products derived therefrom. In embodiments, the dehydrogenation catalyst component can cause the olefins or products derived therefrom to undergo transfer dehydrogenation. In embodiments, the dehydrogenation catalyst component can include one or more elements selected from International Union of Pure and Applied Chemistry (IUPAC) groups 5-10. In embodiments, the dehydrogenation catalyst component can comprise platinum, iridium, ruthenium, rhenium, or combinations thereof. In embodiments, the dehydrogenation catalyst component is selected from the group consisting of platinum, iridium, ruthenium, rhenium, and combinations thereof.

In embodiments, the reactor may comprise one or more catalyst compositions that comprise the two or more catalyst components. For instance, in embodiments, a catalyst composition can comprise a metathesis catalyst component and an isomerization catalyst component. In embodiments, a catalyst composition can comprise a dehydrogenation catalyst component and an isomerization catalyst component. In embodiments, a catalyst composition can comprise a metathesis catalyst component and an isomerization catalyst component. In embodiments, a catalyst composition can comprise a dehydrogenation catalyst component and a metathesis catalyst component. In embodiments, a catalyst composition can comprise a dehydrogenation catalyst component, a metathesis catalyst component, and an isomerization catalyst component. In embodiments, a catalyst composition can comprise a dehydrogenation catalyst component, a metathesis catalyst component, or an isomerization catalyst component. In embodiments, the reactor can comprise a first catalyst composition comprising a metathesis catalyst component. In embodiments, the reactor can comprise a second catalyst composition comprising an isomerization catalyst component.

In embodiments, the catalyst composition is designated by a weight percentage of the one or more elements selected from International Union of Pure and Applied Chemistry (IUPAC) groups 5-10. In embodiments, the first catalyst composition can comprise less than or equal to 15 wt. % of any one of the elements selected from the IUPAC groups 5-10 based on the total weight of the first catalyst composition. For instance, in embodiments, the first catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the first catalyst composition. In embodiments, the first catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the first catalyst composition. In embodiments, the first catalyst composition can comprise any one of the elements selected from the IUPAC groups 5-10 in an amount of from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the first catalyst composition.

In embodiments, the second catalyst composition can comprise less than or equal to 15 wt. % of any one of the elements selected from the IUPAC groups 5-10 based on the total weight of the second catalyst composition. For instance, in embodiments, the second catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the second catalyst composition. In embodiments, the second catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the second catalyst composition. In embodiments, the second catalyst composition can comprise any one of the elements selected from the IUPAC groups 5-10 in an amount from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the second catalyst composition.

It should be understood that according to embodiments, the catalyst composition may be made by methods that lead to the desired composition. Some non-limiting instances include incipient wetness impregnation, or vapor phase deposition of metal precursors (either organic or inorganic in nature), followed by their controlled decomposition.

In embodiments, the reactor can be any reactor useful for causing the plastic waste pyrolysis oil to contact the two or more catalyst components in the presence of the alkene reactant and cause the catalytic reactions to proceed. Non-limiting examples of suitable reactors can include a batch reactor, a fixed-bed reactor, a fluidized bed reactor, a continuous stirred tank reactor, a tubular plug flow reactor, a reactive extruder, or combinations thereof. In embodiments two or more reactors can be used, such as two or more reactors in series. In embodiments, the reactor can comprise a reaction zone where the contacting and the catalytic reactions can occur. In embodiments, the two or more catalyst components can be in the same reaction zone. In other embodiments, the reactor can comprise two or more reaction zones. In embodiments, the reactor can include additional processing of the reactants, such as processing of the alkene reactant, the plastic waste pyrolysis oil, and/or the catalyst components. In embodiments, the effluent comprising one or more products from the catalytic reactions can be further processed, such as separation of one or more products from the effluent. For instance, in embodiments, propylene can be separated from the effluent.

In embodiments, a pressure of the alkene reactant in the reactor, such as in the reaction zone during the contacting can be from 0 pounds per square inch gauge (psig) to 3000 psig. For instance, a pressure of the alkene reactant can be of from 0 psig to 3000 psig, from 0 psig to 2000 psig, from 0 psig to 1000 psig, from 0 psig to 900 psig, from 0 psig to 800 psig, from 0 psig to 700 psig, from 0 psig to 600 psig, from 0 psig to 500 psig, or from 100 psig to 3000 psig. In some embodiments, the amount of the alkene reactant used can be quantified by the pressure of the alkene reactant in the reactor. In other embodiments, the amount of the alkene reactant can be quantified by a space velocity of the alkene reactant.

In embodiments, a temperature of the reactor, such as in the reaction zone, during the contacting can be less than or equal to 500° C. For instance, a temperature of the reactor during the contacting can be less than or equal to 450° C., less than or equal to 400° C., less than or equal to 350° C., less than or equal to 300° C., less than or equal to 250° C., or even less than or equal to 200° C. In embodiments, a temperature of the reactor during the contacting can be of from 50° C. to 500° C., from 50° C. to 400° C., from 50° C. to 350° C., from 50° C. to 300° C., from 50° C. to 250° C., from 50° C. to 200° C., from 60° C. to 400° C., from 60° C. to 350° C., from 60° C. to 300° C., from 60° C. to 250° C., or from 60° C. to 200° C. Without intending to be bound by any particular theory, it is believed that a reduced reactor temperature, such as less than or equal to 500° C., less than or equal to 450° C., less than or equal to 400° C., less than or equal to 350° C., less than or equal to 300° C., or less than or equal to 250° C., can reduce the formation of undesired side products during the contacting. Further, the reduced operational temperature of the reactor can reduce the energy required for the process, which can also reduce the economic cost of operating.

In embodiments, the contacting causes at least a portion of the olefins in the plastic waste pyrolysis oil to undergo catalytic reactions to produce an effluent. In embodiments, the effluent can comprise at least the alkene product of chemical formula CₘH₂ₘ. In embodiments, the alkene product is a compound of chemical formula CₘH₂ₘ, where m is an integer from 3 to 20. For instance, the alkene product can be a compound of chemical formula CₘH₂ₘ, where m is an integer from 3 to 15, from 3 to 10, from 3 to 8, from 3 to 7, from 3 to 6, from 3 to 5, from 3 to 4, or of 3. In embodiments, the alkene product can comprise propylene, butenes, pentenes, or combinations thereof. In embodiments, the alkene product can be selected from the group consisting of propylene, butenes, pentenes, and combinations thereof. In embodiments, the alkene product can consist essentially of, or consist of, propylene, butenes, pentenes, or combinations thereof. In embodiments, the alkene product can consist essentially of, or consist of propylene. In embodiments, at least a portion of the alkene product can be separated from the effluent to produce a modified effluent.

In embodiments, the effluent can comprise at least 1 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, or even at least 60 wt. % of the alkene product.

### EXAMPLES

The various aspects of the present disclosure will be further clarified by the following examples. The examples are illustrative in nature and should not be understood to limit the subject matter of the present disclosure.

### Example 1. Purification of Plastic Waste Pyrolysis Oil

In Example 1, plastic waste pyrolysis oil was purified using various filtration methods. Plastic waste pyrolysis oil was placed under nitrogen atmosphere by bubbling nitrogen through the pyrolysis oil for 15 min. In addition, some samples were treated by either passing 20 mL of pyrolysis oil through a fritted filter with a short plug of silica gel (~40 g), basic alumina (~40 g), or placing over F-200 alumina beads (~2 g). The purification method for each example is identified in Table 1. The measured groups of paraffins, olefins, naphthenes, and aromatics of the pyrolysis oil is shown in Table 2

TABLE 1

| Example | Pyrolysis Oil Purification Method |
|---|---|
| Ex. 1A | Untreated |
| Ex. 1B | Silica Gel |
| Ex. 1C | Basic Alumina |
| Ex. 1D | F-200 Alumina Beads |

TABLE 2

| Pyrolysis Oil Components | Composition (%) |
|---|---|
| Paraffins | 14 |
| Olefins | 71 |
| Naphthenes | 3 |
| Aromatics | 12 |
| Total | 100 |

### Example 2. Isomerization of Pyrolysis Oil with Alkene Zipper

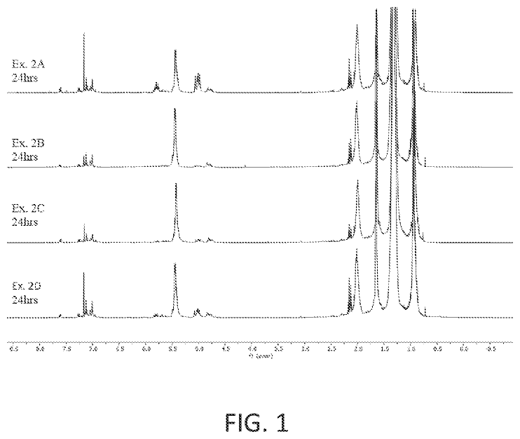

In Example 2, four oven-dried vials were charged with Acetonitrile (cyclopentadienyl)[2-(di-i-propylphosphino)-4-(t-butyl)-1-methyl-1H-imidazole]ruthenium(II) hexafluorophosphate (10 mg), commercially available as Alkene Zipper from Strem Chemicals, Inc., C₆D₆ (0.5 mL), 100 mg of one of the pyrolysis oil samples of Examples 1A, 1B, 1C, and 1D, and a stir-bar, corresponding to Examples 2A, 2B, 2C, and 2D, respectively. The mixture was gently stirred and heated to 200° C. A first portion of the mixture was then extracted with CDCl₃ after 10 minutes, and a second portion was extracted after 24 hours. The resulting solutions were analyzed by ¹H NMR spectroscopy (FIG. 1). The conversion (%) to internal isomers as a function of the purification technique is shown in Table 3.

TABLE 3

| | | Isomerization | Isomerization |
|---|---|---|---|
| | | Conversion (%), 10 | Conversion (%), 24 |
| Example | Pyrolysis Oil | min | hr |
| Ex. 2A | Ex. 1A | 20 | 54 |
| Ex. 2B | Ex. 1B | 35 | 90 |
| Ex. 2C | Ex. 1C | 33 | 88 |
| Ex. 2D | Ex. 1D | 26 | 78 |

### Example 3. Isomerization of Pyrolysis Oil with γ-Alumina

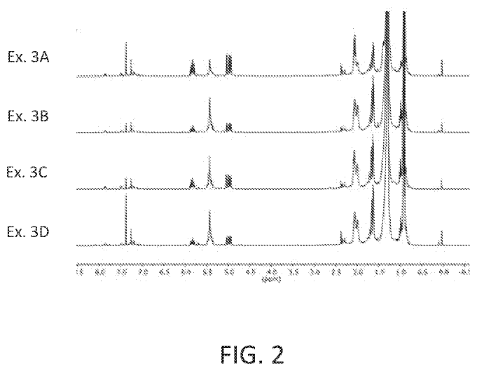

In example 3, γ-Alumina (20 g) was calcined at 500° C. with a ramp rate of 10° C./min for 5 h in dry air, cooled to room temperature, and then transferred into a nitrogen-filled glovebox. Four oven-dried vials were charged with the γ-alumina (200 mg), 400 mg of one of the pyrolysis oil samples of Examples 1A, 1B, 1C, and 1D, and a stir-bar, corresponding to Examples 3A, 3B, 3C, and 3D, respectively. The mixture was gently stirred and heated to 200° C. for 16 hrs. The mixture was then extracted with CDCl₃ and the resulting solution was analyzed by ¹H NMR spectroscopy, as shown in FIG. 2. The conversion (%) to internal isomers as a function of the purification technique is shown in Table 4.

TABLE 4

| | | Isomerization |
|---|---|---|
| Example | Pyrolysis Oil | Conversion (%) |
| Ex. 3A | Ex. 1A | 30 |
| Ex. 3B | Ex. 1B | 67 |
| Ex. 3C | Ex. 1C | 55 |
| Ex. 3D | Ex. 1D | 66 |

### Example 4. Tandem Isomerization and Ethenolysis of Plastic Waste Pyrolysis Oil

In Example 4, the pyrolysis oil (84 g) was passed through a fritted filter packed with silica gel (23 g). The resulting oil was placed under an atmosphere of nitrogen by bubbling nitrogen through the resulting oil for 18 min. The oil was placed in a nitrogen-filled glove box and treated with activated 3 A-mol sieves (6 g) overnight to yield the purified pyrolysis oil. In a nitrogen filled glovebox, a 600 mL Parr reactor (Series 4560, mini bench top reactor) was charged with the purified pyrolysis oil (10 mL), triisopropylbenzene (TIPB, 0.5 mL), and toluene (100 mL). After mixing, an aliquot was taken to determine the initial pyrolysis oil/TIPB ratio by gas chromatography (Agilent 7890A). The reactor was closed off, taken out of the glove box, and heated to 65° C. Once the temperature was stable, Alkene Zipper (35 mg, in 5 mL toluene) was injected into the reaction, and stirred under a nitrogen atmosphere for 0.5 h.

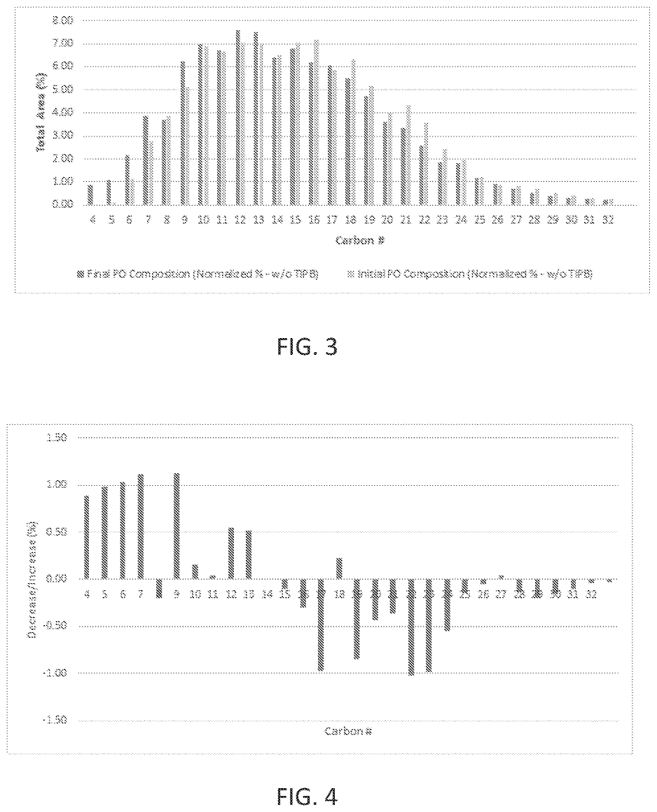

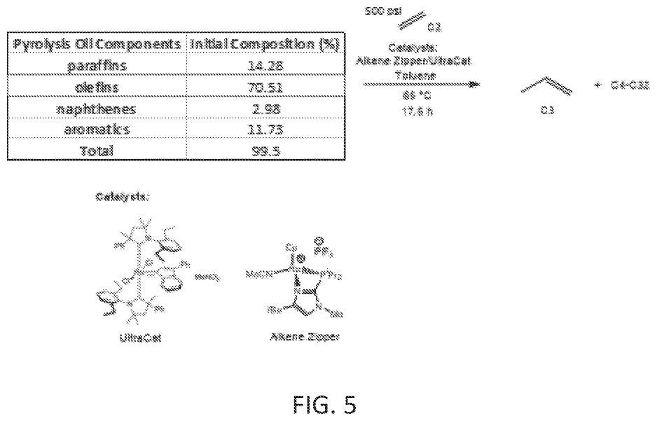

Bis[1-[2,6-diethylphenyl]-3,5,5-trimethyl-3-phenylpyrrolidin-2-ylidene][3-phenyl-1H-inden-1-ylidene]Ru[II]Cl₂, (50 mg, in 5 mL toluene), commercially available as UltraCat from [Strem Chemicals, Inc.], was then added. The reactor was pressurized with ethylene (500 psi), closed off from additional ethylene, and stirred for 17 hours. A gas sample was taken from the headspace at 65° C., using a bomb and Tedlar bag. The sample was analyzed by gas chromatography using a [Agilent 490 Micro GC, confirming formation of propylene (0.5 g). The reactor was cooled (<15° C.), the reactor pressure was relieved, and a solution aliquot was analyzed using a Agilent 7890A GC, confirming a reduction in the overall molecular weight of the pyrolysis oil, as shown in FIGS. 3-4 and Table 5.

TABLE 5
Ex. 4 Final Composition using Gas Chromatography

| | Initial Normalized | Final Normalized | |
|---|---|---|---|
| Carbon | Total | Total | |
| # | Area (% w/o TIPB) | Area (% w/o TIPB) | Change (%) |
| 4 | 0.00 | 0.89 | 0.89 |
| 5 | 0.10 | 1.07 | 0.98 |
| 6 | 1.10 | 2.14 | 1.03 |
| 7 | 2.74 | 3.86 | 1.12 |
| 8 | 3.88 | 3.69 | −0.20 |
| 9 | 5.11 | 6.23 | 1.12 |
| 10 | 6.87 | 7.02 | 0.15 |
| 11 | 6.67 | 6.71 | 0.04 |
| 12 | 7.06 | 7.61 | 0.55 |
| 13 | 7.01 | 7.52 | 0.51 |
| 14 | 6.49 | 6.38 | −0.10 |
| 15 | 7.06 | 6.77 | −0.30 |
| 16 | 7.16 | 6.19 | −0.98 |
| 17 | 5.83 | 6.05 | 0.23 |
| 18 | 6.33 | 5.48 | −0.85 |
| 19 | 5.16 | 4.73 | −0.43 |
| 20 | 3.99 | 3.63 | −0.36 |
| 21 | 4.36 | 3.33 | −1.03 |
| 22 | 3.59 | 2.60 | −0.99 |
| 23 | 2.41 | 1.85 | −0.55 |
| 24 | 1.95 | 1.80 | −0.15 |
| 25 | 1.20 | 1.15 | −0.05 |
| 26 | 0.89 | 0.92 | 0.04 |
| 27 | 0.83 | 0.69 | −0.14 |
| 28 | 0.70 | 0.50 | −0.20 |
| 29 | 0.54 | 0.38 | −0.15 |
| 30 | 0.40 | 0.30 | −0.11 |
| 31 | 0.31 | 0.26 | −0.04 |
| 32 | 0.25 | 0.22 | −0.03 |

### Example 5. Tandem Isomerization and Metathesis of Plastic Waste Pyrolysis Oil and 1-butene Using UltraNitroCat and Alkene Zipper Catalyst

In Example 5, a 600 mL Parr reactor (Series 4560, mini bench top reactor) was charged, in a nitrogen filled glovebox, with the purified pyrolysis oil (Ex. 5A, 4.0 mL), triisopropylbenzene (TIPB, 0.5 mL), and methylcycloxane (20 mL). Acetonitrile(cyclopentadienyl)[2-(di-i-propylphosphino)-4-(t-butyl)-1-methyl-1H-imidazole]ruthenium(II)hexafluorophosphate, commercially available as Alkene Zipper Catalyst from [Strem Chemicals, Inc.](44 mg, in 5 mL MCH), and (1-(2,6-diethylphenyl)-3,5,5-trimethyl-3-phenylpyrrolidin-2-ylidene)dichloro (2-isopropoxy-5-nitrobenzylidene)ruthenium(II) commercially available as UltraNitroCat from [Strem Chemicals, Inc.](50 mg, in 5 mL methylcycloxane) were added into the reactor. Cold (−35° C.) 1-butene (2.57 g, 0.046 mol) was added and the reactor was closed off, taken out of the glove box, heated to 65° C., and stirred at 500 rpm for 16 h. The reactor was cooled (~10° C.), and a gas sample was taken from the headspace using a bomb and a Tedlar bag. The sample was analyzed by gas chromatography using a [Agilent 490 Micro GC], confirming formation of ethylene (50 mol %), propylene (31 mol %), and butenes (19 mol %) in the headspace. A 0.5 mL aliquot (Ex. 5B) was taken from the Parr reactor placed in a standard GC vial. Additional UltraNitroCat (50 mg, in 5 mL methylcycloxane) from [Strem Chemicals, Inc.] were added into the reactor and the reactor was closed off, heated to 65° C., and stirred at 500 rpm for an additional 16 h. The reactor was cooled (~10° C.), and a gas sample was taken from the headspace using a bomb and a Tedlar bag. The sample was analyzed by gas chromatography using a [Agilent 490 Micro GC], confirming formation of ethylene (34 mol %, propylene 35 mol %, and butene (32 mol %) in the headspace. A 0.5 mL aliquot (Ex. 5C) was taken from the Parr reactor placed in a standard GC vial. The composition of the GC vial samples was analyzed as-is with a GC-VUV instrument using an automated PIONA class and multi-parameter analysis with a modified ASTM D8071A method and is reported in Tables 6-8.

TABLE 6
Weight % (Wt %) Composition of Ex. 5A,
Ex. 5B, Ex. 5C. (Obtained with GC-VUV).

| Components | Ex. 5A Wt % | Ex. 5B Wt % | Ex. 5C Wt % |
|---|---|---|---|
| Paraffin | 17.4 | 18.7 | 14.8 |
| Isoparaffin | 2.9 | 2.9 | 7.3 |
| Total Olefin | 64.3 | 66.4 | 67.1 |
| Olefin (C2-C6) | 1.2 | 19.7 | 33.9 |
| Olefin (C7-C20+) | 63.2 | 46.7 | 33.2 |
| Naphthene | 0.4 | 0.2 | 0.1 |
| Aromatic | 14.9 | 11.9 | 10.7 |

TABLE 7
Weight % (Wt %) Composition of
Ex. 5A (Obtained with GC-VUV).

| Carbon. | Paraffin | Isoparaffin | Olefin | Naphthene | Aromatic |
|---|---|---|---|---|---|
| No. | (Wt %) | (Wt %) | (Wt %) | (Wt %) | (Wt %) |
| C2 | 0.00 | 0.00 | 0.00 | 0.00 | 0.00 |
| C3 | 0.00 | 0.00 | 0.00 | 0.00 | 0.00 |
| C4 | 0.00 | 0.00 | 0.00 | 0.00 | 0.00 |
| C5 | 0.00 | 0.00 | 0.00 | 0.00 | 0.00 |
| C6 | 0.23 | 0.10 | 1.18 | 0.00 | 0.00 |
| C7 | 0.80 | 0.00 | 3.04 | 0.00 | 2.72 |
| C8 | 1.20 | 1.46 | 3.68 | 0.00 | 3.00 |
| C9 | 1.37 | 0.04 | 5.80 | 0.04 | 0.99 |
| C10 | 1.40 | 0.13 | 8.62 | 0.06 | 5.73 |
| C11 | 1.76 | 0.25 | 7.76 | 0.22 | 0.79 |
| C12 | 1.81 | 0.27 | 6.51 | 0.00 | 0.44 |
| C13 | 1.44 | 0.30 | 6.12 | 0.12 | 0.00 |
| C14 | 1.70 | 0.32 | 5.81 | 0.00 | 1.25 |
| C15 | 1.57 | 0.00 | 4.74 | 0.00 | 0.02 |
| C16 | 1.31 | 0.00 | 3.50 | 0.00 | 0.00 |
| C17 | 0.95 | 0.00 | 2.70 | 0.00 | 0.00 |
| C18 | 0.73 | 0.00 | 1.55 | 0.00 | 0.00 |
| C19 | 0.55 | 0.00 | 1.41 | 0.00 | 0.00 |
| C20+ | 0.56 | 0.00 | 1.91 | 0.00 | 0.00 |
| Total | 17.4 | 2.9 | 64.3 | 0.4 | 14.9 |

0 = None detected

TABLE 8
Weight % (Wt %) Composition of
Ex. 5B (Obtained with GC-VUV).

| Carbon. | Paraffin | Isoparaffin | Olefin | Naphthene | Aromatic |
|---|---|---|---|---|---|
| No. | (Wt %) | (Wt %) | (Wt %) | (Wt %) | (Wt %) |
| C2 | 0.00 | 0.00 | 0.00 | 0.00 | 0.00 |
| C3 | 0.00 | 0.00 | 0.00 | 0.00 | 0.00 |
| C4 | 0.00 | 0.00 | 6.43 | 0.00 | 0.00 |
| C5 | 0.00 | 0.00 | 3.55 | 0.00 | 0.00 |
| C6 | 0.09 | 0.06 | 9.73 | 0.00 | 0.00 |
| C7 | 0.50 | 0.00 | 0.85 | 0.00 | 1.64 |
| C8 | 0.90 | 2.03 | 1.67 | 0.00 | 1.38 |
| C9 | 1.19 | 0.00 | 3.13 | 0.06 | 0.66 |
| C10 | 1.42 | 0.07 | 4.61 | 0.03 | 5.69 |
| C11 | 1.99 | 0.15 | 5.49 | 0.00 | 0.83 |
| C12 | 1.92 | 0.23 | 6.11 | 0.00 | 0.55 |
| C13 | 1.62 | 0.16 | 5.14 | 0.09 | 0.00 |
| C14 | 1.70 | 0.16 | 4.98 | 0.00 | 1.07 |
| C15 | 1.96 | 0.00 | 3.61 | 0.00 | 0.09 |
| C16 | 1.58 | 0.00 | 3.38 | 0.00 | 0.00 |
| C17 | 1.23 | 0.00 | 2.90 | 0.00 | 0.00 |
| C18 | 0.93 | 0.00 | 1.49 | 0.00 | 0.00 |
| C19 | 0.84 | 0.00 | 1.35 | 0.00 | 0.00 |
| C20+ | 0.81 | 0.00 | 1.92 | 0.00 | 0.00 |
| Total | 18.68 | 2.87 | 66.37 | 0.19 | 11.90 |

0 = None detected

TABLE 9
Weight % (Wt %) Composition of
Ex. 5C. (Obtained with GC-VUV).

| Carbon. | Paraffin | Isoparaffin | Olefin | Naphthene | Aromatic |
|---|---|---|---|---|---|
| No. | (Wt %) | (Wt %) | (Wt %) | (Wt %) | (Wt %) |
| C2 | 0.00 | 0.00 | 0.00 | 0.00 | 0.00 |
| C3 | 0.00 | 0.00 | 0.00 | 0.00 | 0.00 |
| C4 | 0.00 | 0.00 | 8.71 | 0.00 | 0.00 |
| C5 | 0.00 | 0.00 | 7.12 | 0.00 | 0.00 |
| C6 | 0.37 | 0.42 | 18.08 | 0.00 | 0.00 |
| C7 | 0.57 | 0.00 | 0.49 | 0.00 | 2.89 |
| C8 | 1.03 | 5.24 | 1.76 | 0.00 | 1.53 |
| C9 | 1.20 | 0.17 | 2.91 | 0.00 | 0.22 |
| C10 | 1.31 | 0.00 | 3.59 | 0.04 | 5.28 |
| C11 | 1.56 | 0.42 | 4.00 | 0.00 | 0.74 |
| C12 | 1.81 | 0.26 | 4.84 | 0.00 | 0.03 |
| C13 | 1.08 | 0.46 | 3.86 | 0.06 | 0.00 |
| C14 | 1.41 | 0.30 | 2.96 | 0.00 | 0.00 |
| C15 | 1.51 | 0.00 | 2.30 | 0.00 | 0.00 |
| C16 | 1.18 | 0.00 | 2.24 | 0.00 | 0.00 |
| C17 | 0.84 | 0.00 | 1.73 | 0.00 | 0.00 |
| C18 | 0.54 | 0.00 | 1.09 | 0.00 | 0.00 |
| C19 | 0.44 | 0.00 | 0.79 | 0.00 | 0.00 |
| C20+ | 0.00 | 0.00 | 0.63 | 0.00 | 0.00 |
| Total | 14.84 | 7.28 | 67.09 | 0.11 | 10.68 |

0 = None detected

### Example 6. Tandem Isomerization and Metathesis of Plastic Waste Pyrolysis Oil with Grubbs Catalyst® M204 and Alkene Zipper Catalyst

In Example 6, a 600 mL Parr reactor (Series 4560, mini bench top reactor) was charged, in a nitrogen filled glovebox, with the purified pyrolysis oil (Ex. 5A, 4.0 mL), triisopropylbenzene (TIPB, 0.5 mL), and methylcycloxane (20 mL). Acetonitrile(cyclopentadienyl)[2-(di-i-propylphosphino)-4-(t-butyl)-1-methyl-1H-imidazole]ruthenium(II) hexafluorophosphate, commercially available as Alkene Zipper Catalyst from [Strem Chemicals, Inc.](44 mg, in 5 mL MCH), and Benzylidene[1,3-bis(2,4,6-trimethylphenyl)-2-imidazolidinylidene]dichloro (tricyclohexylphosphine)rutheniumum, commercially available as Grubbs Catalyst® M204 from [Sigma Aldrich, Inc.](50 mg, in 5 mL methylcycloxane) were added into the reactor. Cold (−35° C.) 1-butene (2.57 g, 0.046 mol) was added and the reactor was closed off, taken out of the glove box, heated to 65° C., and stirred at 500 rpm for 16 h. The reactor was cooled (~10° C.), no headspace sample was taken due to lack of pressure. A 0.5 mL aliquot (Ex. 6B) was taken from the Parr reactor placed in a standard GC vial. The composition of the GC vial samples was analyzed as-is with a GC-VUV instrument using an automated PIONA class and multi-parameter analysis with a modified ASTM D8071A method and is reported in Tables 10 and 11.

TABLE 10
Weight % (Wt %) Composition of
Ex. 6B. (Obtained with GC-VUV).

| | Ex. 5A | Ex. 6B |
|---|---|---|
| Components | (Wt %) | (Wt %) |
| Paraffin | 17.4 | 11.8 |
| Isoparaffin | 2.9 | 3.1 |
| Total Olefin | 64.3 | 74.4 |
| Olefin (C2-C6) | 1.2 | 27.0 |
| Olefin (C7-C20+) | 63.2 | 47.3 |
| Naphthene | 0.4 | 0.3 |
| Aromatic | 14.9 | 10.4 |

TABLE 11
Weight % (Wt %) Composition of
Ex. 6B. (Obtained with GC-VUV).

| Carbon. | Paraffin | Isoparaffin | Olefin | Naphthene | Aromatic |
|---|---|---|---|---|---|
| No. | (Wt %) | (Wt %) | (Wt %) | (Wt %) | (Wt %) |
| C4 | 0.00 | 0.00 | 0.00 | 0.00 | 0.00 |
| C5 | 0.00 | 0.00 | 10.79 | 0.00 | 0.00 |
| C6 | 0.27 | 0.26 | 16.24 | 0.00 | 0.00 |
| C7 | 0.74 | 0.00 | 2.76 | 0.00 | 3.09 |
| C8 | 0.95 | 1.99 | 2.06 | 0.00 | 1.93 |
| C9 | 0.96 | 0.00 | 2.68 | 0.03 | 0.44 |
| C10 | 1.04 | 0.00 | 2.88 | 0.01 | 3.84 |
| C11 | 1.19 | 0.10 | 2.91 | 0.20 | 0.53 |
| C12 | 1.12 | 0.12 | 3.26 | 0.00 | 0.16 |
| C13 | 0.75 | 0.49 | 2.54 | 0.04 | 0.00 |
| C14 | 0.94 | 0.12 | 2.28 | 0.00 | 0.41 |
| C15 | 1.13 | 0.00 | 1.67 | 0.00 | 0.02 |
| C16 | 0.91 | 0.00 | 1.46 | 0.00 | 0.00 |
| C17 | 0.69 | 0.00 | 1.25 | 0.00 | 0.00 |
| C18 | 0.49 | 0.00 | 0.68 | 0.00 | 0.00 |
| C19 | 0.40 | 0.00 | 0.51 | 0.00 | 0.00 |
| C20+ | 0.26 | 0.00 | 20.40 | 0.00 | 0.00 |
| Total | 11.85 | 3.08 | 74.37 | 0.28 | 10.42 |

0 = None detected

### Example 7. Reduction of Cobalt Oxide-Molybdenum Oxide on Alumina with Hydrogen at 500° C.

In Example 7, cobalt oxide-molybdenum oxide on alumina (CoO/MoO₃/Al₂O₃, 3.5% CoO, 14% MoO3) (20 g), commercially available from Strem Chemicals, Inc was reduced in a quartz tube using a Carbolite VST 12/400 Vertical Split Tube Furnace at 500° C. (w/a 10° C. ramp rate) for 5 h with a 5% H2/N2 gas mixture. The sample was cooled under nitrogen, closed off and placed in a nitrogen filled glovebox.

### Example 8. Tandem Isomerization and Metathesis of Plastic Waste Pyrolysis Oil with MoO₃CoO-Al₂O₃

In Example 8, a 600 mL Parr reactor (Series 4560, mini bench top reactor) was charged, in a nitrogen filled glovebox, with the plastic waste pyrolysis oil (4.0 mL), triisopropylbenzene (TIPB, 0.5 mL), methylcycloxane (20 mL), and Ex7. (4 g), commercially available from Strem Chemicals, Inc were added into the reactor. Cold (−35° C.) 1-butene (2.57 g, 0.046 mol) was added and the reactor was closed off, taken out of the glove box, heated to 65° C., and stirred at 500 rpm for 16 h. The reactor was cooled (~10° C.), and a gas sample was taken from the headspace using a bomb and a Tedlar bag. The sample was analyzed by gas chromatography using a [Agilent 490 Micro GC], confirming formation of ethylene (52 mol %), propylene (32 mol %), butene (15 mol %) in the headspace. A 0.5 mL aliquot (Ex. 8A) was taken from the Parr reactor placed in a standard GC vial. The composition of the GC vial samples was analyzed as-is with a GC-VUV instrument using an automated PIONA class and multi-parameter analysis with a modified ASTM D8071A method and is reported in Tables 12 and 13.

TABLE 12
Weight % (Wt %) Composition of
Ex. 8A. (Obtained by GC-VUV).

| | Ex. 5A | Ex. 8A |
|---|---|---|
| Components | (Wt %) | (Wt %) |
| Paraffin | 17.4 | 17.2 |
| Isoparaffin | 2.9 | 2.2 |
| Total Olefin | 64.3 | 70.7 |
| Olefin (C2-C6) | 1.2 | 39.4 |
| Olefin (C7-C20+) | 63.2 | 31.3 |
| Naphthene | 0.4 | 0.1 |
| Aromatic | 14.9 | 9.9 |

TABLE 13
Weight % (Wt %) Composition of
Ex. 8A. (Obtained by GC-VUV).

| Carbon. | Paraffin | Isoparaffin | Olefin | Naphthene | Aromatic |
|---|---|---|---|---|---|
| No. | (Wt %) | (Wt %) | (Wt %) | (Wt %) | (Wt %) |
| C4 | 0.24 | 0.00 | 37.04 | 0.00 | 0.00 |
| C5 | 0.00 | 0.00 | 0.78 | 0.00 | 0.00 |
| C6 | 0.25 | 0.11 | 1.55 | 0.00 | 0.02 |
| C7 | 0.83 | 0.00 | 3.05 | 0.00 | 3.09 |
| C8 | 1.09 | 1.46 | 4.53 | 0.00 | 2.06 |
| C9 | 1.10 | 0.00 | 4.14 | 0.03 | 0.44 |
| C10 | 1.23 | 0.04 | 5.11 | 0.02 | 3.64 |
| C11 | 1.40 | 0.24 | 4.26 | 0.00 | 0.49 |
| C12 | 1.93 | 0.11 | 2.76 | 0.00 | 0.14 |
| C13 | 1.11 | 0.09 | 2.84 | 0.06 | 0.00 |
| C14 | 1.33 | 0.14 | 2.09 | 0.00 | 0.00 |
| C15 | 1.76 | 0.00 | 1.01 | 0.00 | 0.01 |
| C16 | 1.53 | 0.00 | 0.80 | 0.00 | 0.00 |
| C17 | 1.21 | 0.00 | 0.40 | 0.00 | 0.00 |
| C18 | 0.86 | 0.00 | 0.22 | 0.00 | 0.00 |
| C19 | 0.66 | 0.00 | 0.11 | 0.00 | 0.00 |
| C20+ | 0.61 | 0.00 | 0.00 | 0.00 | 0.00 |
| Total | 17.15 | 2.19 | 70.67 | 0.10 | 9.89 |

0 = None detected

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter of the present disclosure has been described in detail and by reference to specific embodiments. It should be understood that any detailed description of a component or feature of one or more embodiments does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A process for converting olefins in plastic waste pyrolysis oil to at least an alkene product of chemical formula CₘH₂ₘ, the process comprising contacting the plastic waste pyrolysis oil with two or more catalyst components in a reactor, the reactor comprising an alkene reactant of chemical formula CₙH₂ₙ;
where:
m is an integer from 3 to 20 n is an integer from 2 to 20;
the two or more catalyst components comprise a metathesis catalyst component and an isomerization catalyst component; and
contacting causes at least a portion of the olefins in the plastic waste pyrolysis oil, or products derived therefrom, to undergo metathesis reactions and isomerization reactions to produce an effluent comprising at least the alkene product of chemical formula CₘH₂ₘ.

2. The process of claim 1, wherein a pressure of the alkene reactant in the reactor during the contacting is from 0 pounds per square inch gauge (psig) to 3000 psig.

3. The process of claim 1, wherein a temperature of the reactor during the contacting is less than or equal to 500° C.

4. The process of claim 1, wherein the alkene reactant comprises ethylene, propylene, butenes, pentenes, or combinations thereof.

5. The process of claim 1, wherein the alkene product comprises propylene, butenes, pentenes, or combinations thereof.

6. The process of claim 1, wherein the plastic waste pyrolysis oil has a density of from 0.64 g/cm³ to 0.95 g/cm³.

7. The process of claim 1, wherein the plastic waste pyrolysis oil comprises from 20 weight percent (wt. %) to 100 wt. % olefins.

8. The process of claim 1, wherein the plastic waste pyrolysis oil is purified before the contacting.

9. The process of claim 1, wherein the metathesis catalyst component comprises an element selected from International Union of Pure and Applied Chemistry (IUPAC) groups 5-10.

10. The process of claim 1, wherein the metathesis catalyst component comprises rhenium, ruthenium, tungsten, molybdenum, vanadium or combinations thereof.

11. The process of claim 1, wherein the isomerization catalyst component comprises an element selected from International Union of Pure and Applied Chemistry (IUPAC) groups 5-10.

12. The process of claim 1, wherein the isomerization catalyst component comprises alumina, silica, iridium, palladium, ruthenium or combinations thereof.

13. The process of claim 1, wherein the process comprises contacting the plastic waste pyrolysis oil with three or more catalyst components, and wherein the three or more catalyst components comprise a metathesis catalyst component, an isomerization catalyst component, and a dehydrogenation catalyst component.

14. The process of claim 13, wherein the dehydrogenation catalyst component comprises comprises an element selected from International Union of Pure and Applied Chemistry (IUPAC) groups 5-10.

15. The process of claim 1, further comprising separating at least a portion of the alkene product from the effluent.
